SEQUENCE LISTING (44 sequences)

SEQ ID NO: 1 - DNA, 30 bp, Artificial sequence
  Description of the artificial sequence oligonucleotide
gcagcatatgagccatattcaacgggaaac

SEQ ID NO: 2 - DNA, 29 bp, Artificial sequence
  Description of the artificial sequence oligonucleotide
gcagcatatgcaacgggaaacgtcgaggc

SEQ ID NO: 3 - DNA, 31 bp, artificial sequence
  Description of the artificial sequence oligonucleotide
gcagcatatgacgtcgaggccgcgattaaat

SEQ ID NO: 4 - DNA, 31 bp, artificial sequence
  Description of the artificial sequence oligonucleotide
gcagcatatgccgcgattaaattccaacatg

SEQ ID NO: 5 - DNA, 30 bp, Artificial sequence
  Description of the artificial sequence oligonucleotide
gcagcatatgaattccaacatggatgctga

SEQ ID NO: 6 - DNA, 31 bp, Artificial sequence
  Description of the artificial sequence oligonucleotide
gcagcatatgatggatgctgatttatatggg

SEQ ID NO: 7 - DNA, 32 bp, Artificial sequence
  Description of the artificial sequence oligonucleotide
gcagcatatggatttatatgggtataaatggg

SEQ ID NO: 8 - DNA, 29 bp, Artificial sequence
  Description of the artificial sequence oligonucleotide
gcagcatatggggtataaatgggctcgcg

SEQ ID NO: 9 - DNA, 29 bp, Artificial sequence
  Description of the artificial sequence oligonucleotide
gcagcatatgtgggctcgcgataatgtcg

SEQ ID NO: 10 - DNA, 29 bp, Artificial sequence
  Description of the artificial sequence oligonucleotide
gcagcatatggataatgtcgggcaatcag

SEQ ID NO: 11 - DNA, 31 bp, Artificial sequence
  Description of the artificial sequence oligonucleotide
gcagcatatggggcaatcaggtgcgacaatc

SEQ ID NO: 12 - DNA, 31 bp, Artificial sequence
  Description of the artificial sequence oligonucleotide
gcagcatatgggtgcgacaatctatcgcttg

SEQ ID NO: 13 - DNA, 31 bp, Artificial sequence
  Description of the artificial sequence oligonucleotide
gcagcatatgatctatcgcttgtatgggaag

SEQ ID NO: 14 - DNA, 29 bp, Artificial sequence
  Description of the artificial sequence oligonucleotide
gcagcatatgttgtatgggaagcccgatg

SEQ ID NO: 15 - DNA, 28 bp, Artificial sequence
  Description of the artificial sequence oligonucleotide
gcagcatatgaagcccgatgcgccagag

SEQ ID NO: 16 - DNA, 28 bp, Artificial sequence
  Description of the artificial sequence oligonucleotide
gcagcatatggcgccagagttgtttctg

SEQ ID NO: 17 - DNA, 31 bp, Artificial sequence
  Description of the artificial sequence oligonucleotide
gcagcatatggttgtttctgaaacatggcaa

SEQ ID NO: 18 - DNA, 29 bp, Artificial sequence
  Description of the artificial sequence oligonucleotide
gcagcatatggaaacatggcaaaggtagc

SEQ ID NO: 19 - DNA, 30 bp, Artificial sequence
  Description of the artificial sequence oligonucleotide
gcagcatatgcaaaggtagcgttgccaatg

SEQ ID NO: 20 - DNA, 30 bp, Artificial sequence
  Description of the artificial sequence oligonucleotide
gcagcatatgcgttgccaatgatgttacag

SEQ ID NO: 21 - DNA, 32 bp, Artificial sequence
  Description of the artificial sequence oligonucleotide
cgtgctcgagttagaaaaactcatcgagcatc

SEQ ID NO: 22 - DNA, 34 bp, Artificial sequence
  Description of the artificial sequence oligonucleotide
cgtgctcgagttaatcgagcatcaaatgaaactg

SEQ ID NO: 23 - DNA, 35 bp, Artificial sequence
  Description of the artificial sequence oligonucleotide
cgtgctcgagttacaaatgaaactgcaatttattc

SEQ ID NO: 24 - DNA, 34 bp, Artificial sequence
  Description of the artificial sequence oligonucleotide
cgtgctcgagttactgcaatttattcatatcagg

SEQ ID NO: 25 - DNA, 36 bp, Artificial sequence
  Description of the artificial sequence oligonucleotide
cgtgctcgagttaattcatatcaggattatcaatac

SEQ ID NO: 26 - DNA, 37 bp, Artificial sequence
  Description of the artificial sequence oligonucleotide
cgtgctcgagttaaggattatcaataccatatttttg

SEQ ID NO: 27 - DNA, 36 bp, Artificial sequence
  Description of the artificial sequence oligonucleotide
cgtgctcgagttaaataccatatttttgaaaaagcc

SEQ ID NO: 28 - DNA, 34 bp, Artificial sequence
  Description of the artificial sequence oligonucleotide
cgtgctcgagttatttttgaaaaagccgtttctg

SEQ ID NO: 29 - DNA, 34 bp, Artificial sequence
  Description of the artificial sequence oligonucleotide
cgtgctcgagttaaagccgtttctgtaatgaagg

SEQ ID NO: 30 - DNA, 34 bp, Artificial sequence
  Description of the artificial sequence oligonucleotide
cgtgctcgagttactgtaatgaaggagaaaactc

SEQ ID NO: 31 - DNA, 32 bp, Artificial sequence
  Description of the artificial sequence oligonucleotide
cgtgctcgagttaaggagaaaactcaccgagg

SEQ ID NO: 32 - DNA, 34 bp, Artificial sequence
  Description of the artificial sequence oligonucleotide
cgtgctcgagttagcagttccataggatggcaag

SEQ ID NO: 33 - DNA, 34 bp, Artificial sequence; identical to SEQ ID NO: 32
  Description of the artificial sequence oligonucleotide

SEQ ID NO: 34 - DNA, 32 bp, Artificial sequence
  Description of the artificial sequence oligonucleotide
cgtgctcgagttataggatggcaagatcctgg

SEQ ID NO: 35 - DNA, 33 bp, Artificial sequence
  Description of the artificial sequence oligonucleotide
cgtgctcgagttaaagatcctggtatcggtctg

SEQ ID NO: 36 - DNA, 32 bp, Artificial sequence
  Description of the artificial sequence oligonucleotide
cgtgctcgagttagtatcggtctgcgattccg

SEQ ID NO: 37 - DNA, 31 bp, Artificial sequence
  Description of the artificial sequence oligonucleotide
cgtgctcgagttatgcgattccgactcgtcc

SEQ ID NO: 38 - DNA, 33 bp, Artificial sequence
  Description of the artificial sequence oligonucleotide
cgtgctcgagttagactcgtccaacatcaatac

SEQ ID NO: 39 - DNA, 38 bp, Artificial sequence
  Description of the artificial sequence oligonucleotide
cgtgctcgagttaaacatcaatacaacctattaatttc

SEQ ID NO: 40 - DNA, 34 bp, Artificial sequence
  Description of the artificial sequence oligonucleotide
cgtgctcgagttaacaacctattaatttcccctc

SEQ ID NO: 41 - DNA, 35 bp, Artificial sequence
  Description of the artificial sequence oligonucleotide
gcacggtaccgaagacaccggcgcaggaacactgc

SEQ ID NO: 42 - DNA, 36 bp, Artificial sequence
  Description of the artificial sequence oligonucleotide
gcacgagctcgaagacgcgccggttgcattcgattc

SEQ ID NO: 43 - DNA, 810 bp, Artificial sequence
  Description of the artificial sequence synthetic gene
atgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 44 - protein, 269 aa, Artificial sequence
  Description of the artificial sequence aminoglycoside-3′-phosphotransferase, Type II
MSHIQRETSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

(19) United States
(12) Patent Application Publication
(10) Pub. No.: US 2014/0066334 A1
(43) Pub. Date: Mar. 6, 2014

(54) METHOD FOR THE PRODUCTION OF READING-FRAME-CORRECT FRAGMENT LIBRARIES

(71) Applicants: Christian Kranz, Regensburg (DE); Michael Liss, Regensburg (DE)

(72) Inventors: Christian Kranz, Regensburg (DE); Michael Liss, Regensburg (DE)

(73) Assignee: GENEART AG, Regensburg (DE)

(21) Appl. No.: 13/997,665

(22) PCT Filed: Dec. 20, 2011
(86) PCT No.: PCT/EP2011/073373

§ 371 Date: Nov. 15, 2013

(30) Foreign Application Priority Data
Dec. 24, 2010 (DE) 10 2010 056 289.0

(51) Int. Cl. C12Q 1/68

(52) U.S. Cl. CPC C12Q 1/6876
USPC 506/16; 506/26

(57) ABSTRACT

The present invention relates to reading-frame-correct fragment libraries, methods for their production, and the use of the fragment libraries for selection of functional polypeptide variants with improved properties.

The present invention relates to fragment libraries, methods for their production, and the use of fragment libraries for the selection of functional polypeptide variants with improved properties.

Up to the present day, the genomes of more than 180 organisms have been completely sequenced. As early as 2001, a first version of the humane genome was published by researcher groups, independent of one another (Lander et al., 2001, Nature 409:860-921; Venter et al., 2001, Science 291:1304-1351.1,2).

Aside from the human genome, genomes of numerous human pathogens such as, for example, Bacillus anthracis (Reed at al., 2002, Science 296:2028-2033), Helicobacter pylori (Tomb et al., 1997, Nature 388:539-547) or Haemophilus influenzae (Fleischmann et al., 1995, Science 269:496-512.5) have been completely decoded.

It is expected that numerous pharmaceutically relevant targets are included among the proteins coded by the sequenced genes. In order to make the known sequence information usable, biochemical assays are being developed to functionally characterize the potential targets or to generate starting material for the selection of inhibitors. Furthermore, clarification of the structure of proteins is gaining an ever more important role in the pharmaceutical industry (Little et al., 2005, Genome Res. 15:1759-17666; Bentley et al., 2007, Drug Discov. Today 12:931-938).

A basic prerequisite for the characterization of the protein, in each instance, is its quantitative expression. For this purpose, it should be possible to produce the corresponding protein or protein derivative not only in a sufficient amount, but also in soluble form. Further requirements for a potential drug target are its functionality, stability, the possibility of preparing it in pure form, and its crystallizability. The heterologous expression of the coding gene in a suitable expression system, which is required for this purpose, as well as the subsequent purification of correctly folded recombinant proteins for structural and functional analyses are probably the most critical steps in this connection.

What is called the “Target Data Base (TargetDB) for Structural Biology” (http:/targetdb.pdb.orgi) (Table 1) provides information concerning the status of projects that serve for clarifying the structure of proteins.

TABLE 1
TargetDB Status Statistics
Number of target (“target”) proteins that were filed in the
TargetDB worldwide by “Structural Genomics” centers: 240,071

| | Total | (%) relative | (%) relative | (%) relative | (%) relative |
|---|---|---|---|---|---|
| | number of | to cloned | to expressed | to purified | to crystallized |
| Status | targets | targets | targets | targets | targets |
| Cloned | 163639 | 100.0 | — | — | — |
| Expressed | 117920 | 72.1 | 100.0 | — | — |
| Soluble | 45629 | 27.9 | 38.7 | — | — |
| Purified | 41815 | 25.6 | 35.5 | 100.0 | — |
| Crystallized | 14250 | 8.7 | 12.1 | 34.1 | 100.0 |
| Diffraction | 7504 | 4.6 | 6.4 | 17.9 | 52.7 |
| quality crystals | | | | | |
| Diffraction | 6369 | 3.9 | 5.4 | 15.2 | 44.7 |
| NMR | 2131 | 1.3 | 1.8 | 5.1 | — |
| HSQC | 3848 | 2.4 | 3.3 | 9.2 | — |
| Crystal structure | 5030 | 3.1 | 4.3 | 12.0 | 35.3 |
| NMR structure | 2030 | 1.2 | 1.7 | 4.9 | — |
| In proteinDB | 7287 | 4.5 | 6.2 | 17.4 | 37 |
| Work completed | 28398 | — | — | — | — |
| Test target | 109 | — | — | — | — |
| Others | 10648 | — | — | — | — |

Statistics show that until the beginning of 2010, it was possible to express not quite 28% of the target proteins in solution. It was possible to crystallize less than 10% of the proteins up to now.

In the literature, different approaches are described for improving the solubility of larger or more complex proteins, which are frequently present as insoluble aggregates in what are called inclusion bodies after superexpression in E. coli. Thus, expression at low temperatures, the use of promoters at different strengths, or fusion with soluble tags, such as, for example, hexa-histidine (His6), maltose binding protein (MBP), glutathione-S-transferase (GST) or thioredoxin (Trx), leads to higher yields of soluble target proteins (Esposito and Chatterjee, 2006, Current Opinion in Biotechology 17:353-358). On a DNA level, as well, folding and solubility of proteins can already be influenced. Thus, it was reported that codon harmonization or the use of rare codons at specific transition regions between protein domains can lead to decelerated translation and thereby to improved folding and solubility (US2008076161, Rosano and Ceccarelli, 2009, Microbial Cell Factories 8:41). However, the approaches described above are only conditionally reliable and often suitable only for specific proteins.

Another possibility for circumventing the stated difficulties with the solubility or crystallizability of target proteins is based on the production of functional fragments of these proteins or polypeptides, which were identified by means of structural predictions based on bioinformatics, on the basis of the underlying amino acid sequence. For this purpose, a shortened variant of the target protein is produced, expressed, and then tested for solubility. Often, these shortened derivatives can he expressed and crystallized in solution significantly better than full-length proteins (Prodromou et al., 2007, Drug Discov. Today 12:931-938; Savva et al., 2007, Drug Discov. Today 12:939-947).

However, even rational approaches to structural prediction of shortened protein sequences do not always lead to protein variants with the desired properties, because changes in the amino acid sequence can have a cumulative effect on different aspects of the protein structure. The currently available bioinformatics programs cannot make any precise predictions concerning what modification or shortening will actually improve the solubility of the proteins or other functional properties.

For this reason, variant or fragment libraries are produced, which contain different length variants of the corresponding protein or of the nucleic acid starting sequence that codes for the protein or polypeptide. For this purpose, nucleic acid constructs that code for shortened (or, under some circumstances, also lengthened or mutated) variants of the target protein, are synthesized—e.g. by means of de novo synthesis or amplification of a corresponding partial region of the nucleic add starting sequence by means of a polymerase chain reaction (PCR)-based method. Shortening can take place at only one or also at both ends or termini. The latter can be expedient if a natural terminus interferes with the expression or folding of the protein.

In order not to have to produce every length variant within a fragment library separately, methods are used, for example, that allow simultaneous generation of different fragment lengths. In the state of the art, methods are described in which a nucleic add starting sequence is either enzymatically digested (Campbell and Jackson, 1980, J. Biol. Chem. 5:3726-3735; Melgar and Goldthwait, 1968, J. Biol. Chem. 243:4409-4416; Price, 1972, J. Biol. Chem. 247:2895-2899; Anderson, 1981, Nucleic Acids Res. 9:3015-3027) or physically (e.g. by means of ultrasound) cut (Hengen, 1997, Trends Biochem. Sci. 22:273-274; McKee et al., 1977, Biochemistry 16:4651-4654.14, 15). For enzymatic removal of individual nucleotides at the ends of linear nucleic add sequences, for example, the enzymes exonuclease III or Bal31 are used. (Rogers and Weiss, 1980, Meth. Enzymol. 65:201-211; We et al., 1983, J. Biol. Chem. 258:3506-13512).

In the method called “ITCHY” (“incremental truncation for the creation of hybrid enzymes”) (Ostermeir and Lutz, 2001, Methods in Molecular Biology 231:129-141), a target gene is truncated at one end, in each instance, by means of exonuclease III digestion, while the other end of the gene is fixed in place. After the two ends of the gene have been truncated separately, the diversities that have been formed are combined, for example by means of ligation of the fragments. In another method, random decreases in length are introduced using oligonucleotides, which comprise an unknown variable sequence region that has been fixed in place, in each instance, in the 3′ direction (WO0166798). The variable sequence region of the oligonucleotides hybridizes at suitable regions, and therefore, after PCR-based lengthening of the oligonucleotides (“primer extension”), fragments of the nucleic add starting sequence, with different lengths, are formed.

The methods described above for the production of fragment libraries by means of random shortening of the nucleic add starting sequence have the disadvantage that approximately two-thirds of the resulting fragments have reading frame mutations. Furthermore, the physically cut or uncontrolled enzymatically shortened nucleic add fragments have individual 5′ and/or 3′ overhangs, which must subsequently be treated further enzymatically (e.g. filling up 5′ overhangs with the Klenow fragment of DNA polymerase I or removal of 3′ overhangs with the 3′-5′ exonuclease activity of T4-polymerase), in order to simultaneously allow “blunt end” cloning of all variants into the target vectors, in each instance. Non-directed “blunt end” cloning leads to a further reduction in correct constructs, so that only ⅓×½=⅙ of all fragments are present in the expression vector both in the correct reading frame and in the correct orientation (Prodromou et al., 2007, Drug Discov. Today 12:931-938; Savva et al., 2007, Drug Discov. Today 12:939-947).

A further disadvantage of the methods described in the state of the art is difficult control of the parameters that underlie the decreases in length (enzyme amount, duration of incubation, power of the ultrasound sensor, etc.). As a tendency, it is necessary here to work on multiple batches in parallel, at different parameters, in order to obtain a batch that yields approximately the required results.

A further disadvantage of the methods described is that the central region off the nucleic add starting sequence that is to be shortened at the 5′ and 3′ ends is freely selectable only with effort. Because the full-length construct is shortened, enzymatically or physically, uniformly from both sides, the central region necessarily lies precisely in the middle of the full length. This can only be shifted in that one end of the full-length fragment is first lengthened, for example by means of a portion of the target vector or by means of PCR-based methods.

Another limiting factor is the fact that the 5′ and 3′ decreases in length proceed gradually. It is not possible to undertake deletions in larger steps or only in defined blocks (e.g. by domains). This increases the diversity of the resulting fragment library and the proportion of undesirable Variants and the related screening effort enormously. Furthermore, it is likely that specific deletions are represented more frequently in the fragment library, due to “hot spots” of the exonuclease activity or physically fragile positions that tend to break more easily during cutting.

The task underlying the present invention therefore consists in making available fragment libraries and in an improved method for theft production, wherein the occurrence of nonsense fragments is avoided. In particular, the task consists in producing fragment libraries that contain all the possible combinations of defined length variants with 5′ and 3′ decreases in length, wherein not only the central region but also the exact lengths of the 5′ and 3′ decreases in length and theft distribution in the fragment library can be freely selected.

The task of the underlying invention is accomplished by means of a fragment library comprising at least two, preferably at least four, six, eight, particularly preferably at least 10, 12, 16, 20, 30, 40, 50, 60, 70, 80, 90 or at least 100 length variants of a nucleic add starting sequence, characterized in that
a) each length variant comprises a constant central region and a 5′ and a 3′ region of variable length, in each instance,
b) each length variant is composed at least of a first partial fragment and a second partial fragment, wherein each first partial fragment comprises at least one defined 5′ partial region of the constant central region, and each second partial fragment comprises at least one defined 3′ partial region of the constant central region.

The fragment library is furthermore characterized in that all the first and second partial fragments are connected with one another in a step, in such a manner that the length variants demonstrate all the possible combinations of first and second partial fragments. In a particularly preferred embodiment, all the length variants are represented in the fragment library at approximately the same proportions.

The fragment library according to the invention is furthermore characterized in that it does not contain any nonsense fragments, and that the length variants are composed only of clearly defined first and second partial fragments and do not have any reading frame mutations as compared with the nucleic add starting sequence.

The constant central region is defined in advance and is identical for every length variant. In contrast, the length variants comprise different 5 and/or 3′ regions, the length of which is established by the length of the first and second partial fragments.

The underlying nucleic add starting sequence can be obtained, for example, by means of PCR amplification e.g. from a genomic sequence, by means of cDNA synthesis from cellular mRNAs or by means of restriction digestion of an existing sequence. Alternatively, the nucleic add starting sequence can be generated by de novo synthesis, from synthetic oligonucleotides, in solution or by means of solid-phase synthesis. Corresponding technologies and the underlying molecular biology methods for making a nucleic add starting sequence available are known to a person skilled in the art and are described, for example, in WO03085094, US2003152984, WO9914318, WO02081490, WO0075368, WO0012123, U.S. Pat. No. 4,652,639 or U.S. Pat. No. 5,750,380.

The nucleic add starling sequence can be a wild-type sequence or a modified sequence, or can be composed of wild-type and modified sequences. The modification can consist, for example, in that the nucleic add starting sequence is partially or completely codon-optimized, which means that the codon use for expression in a target organism is adapted to the expression system, in each instance. In addition, motifs can be introduced that modulate expression of the nucleic acid. A method for modulation of gene expression by means of changing the content of iatrogenic Cog dinucleotides is described, for example, in WO2006015789. Not only can sequence motifs that impair expression be removed from the nucleic acid starting sequence, but also sequence motifs that promote expression or other functions can be inserted into it. Thus, for example, computer programs and common algorithms can be used to identify undesirable sequences such as e.g. RNA secondary structures, inhibitory DNA or RNA elements, cryptic splice sites, undesirable restriction cut sites, etc., and to remove them from the nucleic acid starting sequence. If the nucleic acid starting sequence is a sequence that codes for a polypeptide or protein, the changes can be undertaken based on the degeneration of the genetic code, without changing the amino acid sequence. Methods for sequence optimization are described, for example, in WO04059556, WO06015789 or in U.S. Pat. No. 114,148.

Furthermore, for specific purposes (e.g. introduction of additional mutations), not only the nucleic acid starting sequence but also the derived partial fragments or length variants, can contain, along with the common DNA and RNA bases, also modified nucleobases, as described, for example, in Bergstrom, 2009, Curr. Protoc. Nucleic Add Chem. 37:1.4.1-1.4.32.

Furthermore, the nucleic add starting sequence can be coding (for RNA, protein, polypeptide) or non-coding (e.g. comprise an aptamer sequence). It can code for functional RNA such as, for example, rRNA, tRNA, snRNA, mRNA, siRNA, satellite. RNA, a ribozyme or decoy.

Finally, the nucleic add starting sequence can be composed of multiple nucleic adds or comprise untranslated regulatory sequences such as, for example, promoters, transcription or translation signals, enhancer or silencer sequences, introns, ribosomal binding sites or AT-rich inhibitory elements. The untranslated sequences can, however, also take on non-regulatory functions and serve, for example, as space holder sequences.

In a preferred embodiment, at least one part of the nucleic add starting sequence codes for an amino add sequence, wherein the amino add sequence is defined by at least one central region and one amino-terminal and/or one carboxyl-terminal region, and each length variant codes at least one constant central region and one amino-terminal and/or carboxyl-terminal region of variable length, in each instance. In particular cases, a length variant e.g. can have only the constant central region and an amino-terminal region or only the constant central region and a carboxyl-terminal region.

The amino add sequence coded by the nucleic add starting sequence can be a protein, polypeptide or polymer, individual or combined domains or fragments thereof, and derivatives of the said amino add sequences or a fusion protein of homologous or heterologous fusion components.

The amino add sequence can furthermore comprise not only wild-type but also recombinant, chimeric or artificial sequence regions. Furthermore, the amino add sequence can contain not only the natural amino adds but also non-natural amino adds, which can be advantageous for investigation of specific physical, chemical or biological properties of proteins. In bacteria, for example, stop codons can be “reprogrammed” for the inclusion of non-natural amino adds, using new or modified enzymes (Wang et al., 2001, Science 292:498-500). The expansion of the genetic code by means of the inclusion of non-natural amino adds and theft use is described, for example for other in vivo expression systems, in WO04085463, WO10114615, WO09151491, WO09075847, WO08073184, WO08030612 and US2008254540 or for cell-free expression in WO08066583. Alternatively, amino adds can also be modified subsequently (for example chemically) in the translated fragments.

The fragment library according to the invention can comprise at least two, preferably at least four, six, eight, 10, 20, 50, 100, 250, 500, 1000, 10,000, 25,000, 50,000, at least 100.000 or at least 1.000.000 different length variants. The desired diversity of the fragment library can be precisely determined and depends essentially on the length of the nucleic add starting sequence and the length of the constant central region as well as on the question, where, in the case of larger nucleic add starting sequences, the decrease in length can take place not necessarily amino add by amino add, but also in larger steps, for example domain by domain, in order to keep the screening effort low. Also, a “rough frame” fragment library can be produced, at first, where the decreases in length take place in larger steps (by more than three nucleotides or more than one amino acid). After relevant regions have been defined, a further “fine frame” fragment library with decreases in length amino acid by amino add can be derived from these.

In this connection, the length variants of the fragment library, as compared with the nucleic add starting sequence, can have

a shortened 5′ region or

a shortened 3′ region or

a shortened 5′ region and a shortened 3′ region or

a lengthened 5′ region or

a lengthened 3′ region or

a lengthened 5′ region and a lengthened 3′ region, or

a lengthened 5′ region and a shortened 3′ region or

a shortened 5′ region and a lengthened 3′ region or

the fragment library can comprise a partial amount or combination or all possible ones of these length variants.

The fragment library can furthermore contain length variants that have, as compared with the nucleic add starting sequence, insertions or internal deletions or point mutations within the first or second or in the first and second partial fragments.

Finally, the possibility exists of inserting or adding regulatory sequences such as (e.g. promoter/enhancer or terminator sequences, untranslated regions, signal peptide sequences, introns, restriction cut sites, start and stop codons, polyadenylation signal sequences, etc.), which are not contained in the nucleic add starting sequence, into the length variants derived from the nucleic add starting sequence.

The fragment library is furthermore characterized in that it comprises at least two, preferably at least four, particularly preferably at least 10 or at least 100 length variants of a nucleic add starting sequence that comprises a constant central region and a 5′ region that is defined by X nucleotides, and a 3′ region that is defined by Y nucleotides, in each instance, where X and Y are whole numbers and the central region in each length variant is identical, characterized in that
a) the part of the 5′ region derived from the nucleic acid starting sequence, in comparison with the nucleic acid starting sequence, is shortened by X−[m×3] nucleotides, where m is a whole number and equal to or less than X/3, or
b) the part of the 3′ region derived from the nucleic acid starting sequence, in comparison with the nucleic acid starting sequence, is shortened by Y−[n×3] nucleotides, where n is a whole number and equal to or less than Y/3, or
c) the 5 region and the 3′ region are shortened according to a) and b), in each instance.

Furthermore, the fragment library according to the invention can comprise at least two, preferably at least four, six, eight, particularly preferably at least 10, 12, 20, 30, 40, 50, 60, 70, 80, 90 or at least 100 length variants of a nucleic acid starting sequence that codes for an amino add sequence, wherein the amino add sequence comprises a central region, an amino-terminal region coded by X nucleotides and a carboxyl-terminal region coded by Y nucleotides, where X and Y are whole numbers, characterized in that the central region is identical in ever length, and
a) the sequence coding for the amino-terminal region, in comparison with the nucleic add starting sequence, is shortened by X−[m×3] nucleotides, where m is a whole number and equal to or less than X/3 or
b) the sequence coding for the carboxyl-terminal region, in comparison with the nucleic add starting sequence, is shortened by Y−[n×3] nucleotides, where n is a whole number and equal to or less than Y/3 or
c) the sequences coding for the amino-terminal region and the carboxyl-terminal region are shortened according to a) and b), in each instance.

The invention furthermore comprises a method for the production of a fragment library comprising at least two, preferably at least four, six, eight, preferably at least 10, 12, 20, 30, 40, 50, 60, 70, 80, 90 or at least 100 length variants of a nucleic add starting sequence, characterized in that
a) each length variant comprises a constant central region and a 5′ and a 3′ region of variable length, in each instance,
b) each length variant is composed of at least a first partial fragment and a second partial fragment, wherein each first partial fragment comprises at least one defined 5′ partial region of the constant central region and each second partial fragment comprises at least one defined 3′ partial region of the constant central region.

The method is furthermore characterized in that all the first and second partial fragments are connected with one another, in a step, in such a manner that the length variants comprise all the possible combinations of first and second partial fragments. In a preferred embodiment, the partial fragments are combined in such a manner that all the length variants are represented in the fragment library at approximately the same proportions. In a particularly preferred embodiment, the amounts of the first and second partial fragments to be combined can be used in direct proportion to theft length.

In particular cases, a further combination of the first and second partial fragments with third or fourth or further partial fragments can be desirable. In this connection, the third or fourth or further partial fragments can be generated, for example, from a completely different nucleic acid starting sequence or a second nucleic acid starting sequence, modified as compared with the first only in certain partial regions. For example, in this way fragment libraries can be produced from related alleles or protein variants or from different members of a protein family.

Furthermore, the method is characterized in that no combination of first partial fragments with one another or second partial fragments with one another can take place, in order to guarantee uniform distribution of the fragments in the library.

In a preferred embodiment, the first and second partial fragments of the fragment library are produced by way of a PCR-based method, with specific oligonucleotides.

For example, the production of the partial fragments can take place by means of lengthening of oligonucleotides that are homologous with a partial region of the nucleic acid starting sequence, by means of a PCR-based method (“primer extension”), wherein at least approximately 10, better 12, preferably at least 15 nucleotides of the 3 region of the oligonucleotides demonstrate homology or identity with the defined part of the nucleic acid starting sequence of at least approximately 70%, 71%, 72%, 73%, 74%, bevorzugt mindestens 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, oder 100%, and the 5′ region of the oligonucleotides is different from the nucleic acid starting sequence.

Thus, the total length of the oligonucleotide that is homologous with a partial region of the nucleic add starting sequence can comprise approximately 30 nucleotides, but also up to approximately 40 nucleotides. In this connection, for example, the 5′ region of an oligonucleotide that is different from the nucleic add starting sequence, for the production of a first partial fragment, can be structured as follows: a 4 nucleotide overhang is followed by a restriction cut site comprising 6 nucleotides, and by an ATG start codon comprising 3 nucleotides. Thus, in this case, the 5′ region would have a length of 13 nucleotides. The homologous 3′ region, independent of the properties of the non-homologous 5′ region of the oligonucleotide, should be so long that binding to the defined part of the nucleic add starting sequence is possible and PCR-based lengthening of the oligonucleotide can take place.

First, the central region to be kept constant within the nucleic add starting sequence is established. This can be freely defined in terms of length and region. In a preferred embodiment, the central region comprises at least approximately 40 or 45 nucleotides, or 50, 55, 60, 65, 70, 75, 80 or 85 nucleotides, particularly preferably, however, at least 90 or 150 nucleotides. The upper boundary of the constant central region depends on the total length of the nucleic add starting sequence and the underlying problem. In this connection, the nucleic add starting sequence is first divided into two halves, in the center of the previously defined central region, which are referred to as the 5′ and 3′ regions of the nucleic add starting sequence.

In order to produce the first and second partial fragments, a separate oligonucleotide pair is designed for each first and second partial fragment. The oligonucleotide pairs for the production of the first partial fragments each consist of a truncating forward (fwd) oligonucleotide, which binds, with its 3′ end, to a defined sequence in the 5′ region of the nucleic add starting sequence, and a reverse (rev) counter-oligonucleotide, which binds, with its 3′ end, within the constant central region of the nucleic add starting sequence, in the opposite direction. The truncating forward oligonucleotide is varied for the production of the first partial fragments, while the same reverse counter-oligonucleotide is always used. Likewise, the oligonucleotide pairs for the production of the second partial fragments each consists of a truncating reverse oligonucleotide, which binds, with its 3′ end, to a defined sequence in the 3′ region of the nucleic add starting sequence, and a forward counter-oligonucleotide, which binds, with its 3′ end, within the constant central region of the nucleic add starting sequence, in the opposite direction. The truncating reverse oligonucleotide is varied for the production of the second partial fragments, while the same forward counter-oligonucleotide is always used.

In a preferred embodiment, the production of the first partial fragments is characterized by the following steps
a) Making available a defined nucleic acid starting sequence
b) Making available a first oligonucleotide for the synthesis of a first partial fragment, wherein the 3′ region of the first oligonucleotide is identical with a defined nucleic acid sequence in the 5′ region of the nucleic acid starling sequence by at least approximately 70%, 71%, 72%, 73%, 74%, preferably at least 75% or 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%,
c) Making available a second oligonucleotide, wherein the 3′ region of the second oligonucleotide is identical with a defined nucleic add sequence of the constant central region of the nucleic add starting sequence by at least approximately 70%, 71%, 72%, 73%, 74%, preferably at least 75% or 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%,
d) Incubation of the nucleic add starting sequence from a) with the first oligonucleotide from b), the second oligonucleotide from c) and a polymerase system under conditions that are suitable for allowing hybridization of the homologous 3′ regions of the oligonucleotides to single-strand regions of the nucleic add starting sequence and lengthening of the hybridized oligonucleotide by means of polymerase chain reaction,
e) Making available a third oligonucleotide for the synthesis of a further first partial fragment, wherein the 3′ region of the third oligonucleotide is identical with a defined nucleic add sequence in the 5′ region of the nucleic add starting sequence by at least approximately 70%, 71%, 72%, 73%, 74%, preferably at least 75% or 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% and differs in at least one, preferably in at least three nucleotides from the 3 regions of the first oligonucleotide,
f) Incubation of the nucleic add starting sequence from a) with the third oligonucleotide from e), the second oligonucleotide from c) and a polymerase system under conditions that are suitable for allowing hybridization of the homologous 3′ regions of the oligonucleotides to single-strand regions of the nucleic add starting sequence and lengthening of the hybridized oligonucleotides by means of polymerase chain reaction,
g) If necessary, making available further oligonucleotides for the synthesis of further first partial fragments, wherein each 3″ region of each further oligonucleotide is identical with another defined nucleic add sequence in the 5 region of the nucleic add starting sequence by at least approximately 70%, 71%, 72%, 73%, 74%, preferably at least 75% or 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% and differs in at least one, preferably in at least three nucleotides from the 3′ regions of the first and third oligonucleotides,
h) Incubation of the nucleic add starting sequence from a) with a further oligonucleotide from g), the second oligonucleotide from c) and a polymerase system, in each instance, under conditions that are suitable for allowing hybridization of the homologous 3′ regions of the oligonucleotides to the nucleic add starting sequence and its lengthening by means of polymerase chain reaction.
   Repetition of steps g) and h) until all the desired first partial fragments have been synthesized.

Furthermore, in a preferred embodiment, the production of the second partial fragments is characterized by the following steps
a) Making available a defined nucleic add starting sequence
b) Making available a first oligonucleotide for the synthesis of a first partial fragment, wherein the 3′ region of the first oligonucleotide is identical with a defined sequence in the 3′ region of the nucleic add starting sequence by at least approximately 70%, preferably at least 75%,
c) Making available a second oligonucleotide, wherein the 3′ region of the second oligonucleotide is identical with a defined sequence of the constant central region of the nucleic add starting sequence by at least approximately 70%, 71%, 72%, 73%, 74%, preferably at least 75% or 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%,
d) Incubation of the nucleic add starting sequence from a) with the first oligonucleotide from b), the second oligonucleotide from c) and a polymerase system under conditions that are suitable for allowing hybridization of the homologous 3′ regions of the oligonucleotides to single-strand regions of the nucleic add starting sequence and lengthening of the hybridized oligonucleotides by means of polymerase chain reaction,
e) Making available a third oligonucleotide for the synthesis of a further second partial fragment, wherein the 3′ region of the third oligonucleotide is identical with a defined sequence in the 3′ region of the nucleic add starting sequence by at least approximately 70%, 71%, 72%, 73%, 74%, preferably at least 75% or 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% and differs in at least one, preferably three nucleotides from the 3′ regions of the first oligonucleotide,
f) Incubation of the nucleic acid starting sequence from a) with the third oligonucleotide from e), the second oligonucleotide from c) and a polymerase system under conditions that are suitable for allowing hybridization of the homologous 3′ regions of the oligonucleotides to single-strand regions of the nucleic add starting sequence and lengthening of the hybridized oligonucleotides by means of polymerase chain reaction,
g) If necessary, making available further oligonucleotides for the synthesis of further second partial fragment, wherein each 3′ region of each further oligonucleotide is identical with another defined sequence in the 3′ region of the nucleic add starting sequence by at least approximately 70%, 71%, 72%, 73%, 74%, preferably at least 75% or 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% and differs in at least one, preferably at least three nucleotides from the 3′ regions of the first and third oligonucleotides,
h) Incubation of the nucleic add starting sequence from a) with a further oligonucleotide from g), the second oligonucleotide from c) and a polymerase system under conditions that are suitable for allowing hybridization of the homologous 3′ regions of the oligonucleotides to the nucleic add starting sequence and its lengthening by means of polymerase chain reaction.
   Repetition of steps g) and h), until all the desired second partial fragments are synthesized.

In a particularly preferred embodiment of the method for the production of a fragment library, not only all the first but also all the second partial fragments are produced according to the method described, in separate PCR reactions. In this connection, the first and second partial fragments can be produced one after the other or at the same time, for example in parallel PCR reactions.

In a preferred embodiment, the forward and reverse counter-oligonucleotides, in the 5′ region, in each instance, are given a recognition sequence for a TypIIs restriction enzyme that is placed in such a manner that after restriction of the PCR products or the partial fragments with the TypIIs restriction enzyme, a common non-palindromic overhand of optimally at least four nucleotides occurs, which is complementary in the first and second partial fragments and corresponds to part of the central region. By way of the non-palindromic overhangs, the first and second partial fragments generated in separate PCR reactions can be combined.

The invention therefore comprises a method for the production of a fragment library, wherein the production of all first and second partial fragments takes place by means of a PCR-based method, using specific oligonucleotides, wherein at least the forward and reverse counter-oligonucleotides that bind a starting nucleic acid sequence contain recognition sequences, in each instance, for the same or two different TypIIs restriction enzymes, so that the restriction digestion of all first and second partial fragments or a selection thereof, after restriction with the corresponding enzyme(s) leads to generation of single-strand palindromic or preferably non-palindromic overhangs at the 3′ ends of the first and the 5′ ends of the second partial fragments and their subsequent connection in a directed orientation by means of ligation or recombination, so that only connection of first with second partial fragments is possible.

For shortening of the 5° region of the nucleic acid starting sequence, multiple forward oligonucleotides are designed, which bind, with their 3′ end, to a defined sequence of the 5′ regions of the nucleic acid starting sequence, in each instance, in such a manner that the desired first partial fragments with decreases in length of the 5′ region are formed. In this connection, each forward oligonucleotide can append sequences such as a start codon or additional regulatory sequences on its non-hybridizing 5 end (if these are not already present in the target vector) and a cut site for subsequent cloning into the target vector.

Accordingly, multiple reverse oligonucleotides are designed for shortening of the 3′ regions of the nucleic add starting sequence, which bind, with their 3′ end, in each instance, to a defined sequence of the 3′ region of the nucleic add starting sequence, in such a manner that the desired second partial fragments with decreases in length of the 3′ region occur. In this connection, each reverse oligonucleotide can append sequences such as a stop codon or additional regulatory sequences on its non-hybridizing 5′ end (if these are not already present in the target vector), and a cut site for subsequent cloning into the target vector.

In this connection, the forward and reverse oligonucleotides, as a function of the length and/or melting temperature, have a 3′ end, in each instance, that overlaps at least by approximately 12, preferably at least by 13, particularly preferably at least by 18 nucleotides with the binding region of the nucleic add starting sequence that has been established, in each instance, in order to guarantee sufficient hybridization of the oligonucleotides to the 5′ and 3′ regions for the PCR amplification.

In a preferred embodiment, the oligonucleotide pairs have a composition such that step by step shortening of the 5′ and 3′ regions can take place by means of PCR-based lengthening (“primer extension”) of the oligonucleotides hybridized onto the nucleic add starting sequence, in separate PCR reactions. The decreases in length introduced by means of the oligonucleotides take place, in this connection, preferably amino add by amino add, in each instance, or, on a nucleotide level, in steps of three (codon by codon), in order to avoid reading frame jumps in the resulting fragment libraries. As a function of the total length of the nucleic add starting sequence, the decrease in length can also take place, at first, in steps of 6, 9, 12 or even more. A person skilled in the art will consider in advance here what shortening strategy appears practical, depending on the application, and whether entire sections or domains can be skipped, as a function of the problem, in each instance.

In order to obtain a fragment library with defined length variants of the nucleic add starting sequence, the possibility exists of combining all the first with all the second partial fragments or, on the other hand, of making available only a mixture with a selection or partial amount of first or second partial fragments. Also, different mixtures of first and second partial fragments can be produced.

In order to achieve a uniform distribution of the length variants, the concentration of the partial fragments can be precisely measured before they are combined (e.g. photometrically at a wavelength of 260 nm), Then, for example, all or a partial selection of first and second partial fragments, in the desired molar amounts (generally equimolar) are combined. If necessary, however, different amounts of the partial fragment produced separately can also be combined, in order to influence the proportion of the resulting length variants. Thus, in particular cases, it can be advantageous to use larger amounts of longer partial fragments and smaller amounts of shorter partial fragments, in order to avoid preferential formation of shorter length variations.

In a preferred embodiment of the method according to the invention for production of the fragment libraries, the 3 ends of the first partial fragments and the 5′ ends of the second partial fragments have single-strand overhangs, and the first and second partial fragments are connected with one another by means of ligation. The combination of the partial fragments can take place, in this connection, in the presence or absence of ligase or topoisomerase, or alternatively also by means of in vitro recombination or by means of in vivo recombination in the absence of Haase. In this connection, the single-strand overhangs of the first and second partial fragments can be either palindromic, or, in a preferred embodiment, non-palindromic.

In the particularly preferred embodiment, the first and second partial fragments from the mixture obtained are incubated with a suitable enzyme to generate single-strand overhangs on the 3′ ends of the first and the 5′ ends of the second partial fragments, purified, and subsequently connected by means of ligation in the presence of a ligase (e.g. T4 ligase). The enzyme can be an endonuclease such as a TypII restriction endonuclease or, in a preferred embodiment, a TypIIs restriction endonuclease.

The TypIIs restriction enzymes can be, for example, Bbsl, Bsal, BsmBl, Earl, or Ecil. Other suitable TypIIs restriction enzymes can be found, for example, under the following link of the vendor New England Biolabse®: http://www.neb.com/nebecomm/EnzymeFinder.asp?. Particularly preferably, these are restriction enzymes that cut outside of their recognition sequence. In this connection, either all the first and second partial fragments can be digested separately, or the mixture of first and second partial fragments can be digested separately, with different enzymes, or all the first and second partial fragments can be digested in a common step with the same enzyme (“one pot reaction”), in order to produce the single-strand overhang.

Because the overhang generated by means of the TypIIs enzyme is not palindrornic, in a preferred embodiment, only a first partial fragment can ligate with a second partial fragment, in each instance. By means of the ligation, all the possible combinations of first and second partial fragments can be generated, and the diversity is calculated from the product of the number of the first and second partial fragments. Alternatively, the 5′ and 3 ends of the ligated length variants are subsequently cut with further restriction enzymes (e.g. TypII or TypIIs enzymes), in order to generate single-strand overhangs for directed cloning of the length variants into a target vector. This digestion, however, can already take place parallel to the TypIIs digestion of the partial fragments.

To generate the single-strand overhangs of the first and second partial fragments, furthermore an enzyme with exonuclease activity, such as exonuclease III, exonuclease T5, lambda-exonuclease, for example, or such as the exonuclease activity of specific DNA polymerases, for example, can be used. DNA polymerase can be, for example, a T4-DNA polymerase, a T7-DNA polymerase, DNA polymerase I, Klenow DNA polymerase, Pfu polymerase, Phi29-DNA polymerase, Phusion™-High-Fidelity polymerase, VentR®, Deep VentR®, 9°-N. ₘ-DNA polymerase or a Tag polymerase with “proof reading” activity. Corresponding methods are described, for example, in Aslanidis and de Jong, 1990, Nucl. Acids Res. 18:6069, WO07032837, WO09103027, or WO07021944. Because, in the case of exonuclease digestion, also nucleotides in the 3′-5′ direction are removed from the ends of the partial fragments that are not to be fused or are shortened differently, the resulting 5′ overhangs, after combination of the first and second partial fragments, can be filled up again, for example with the Klenow fragment of DNA polymerase I. Alternatively, the resulting 5″ overhangs can serve for cloning of the length variants into a target vector. This possibility can be taken into consideration in the design of the terminal oligonucleotides for the production of the partial fragments.

Alternatively, the single-strand overhangs of the first and second partial fragments can be produced using the USER™ enzyme (mixture of uracil DNA glycosylase (UDG) and DNA glycosylase lyase Endo VIII) from New England Biolabs (see U.S. Pat. No. 7,435,572). For this purpose, a uracil radical is built into the second oligonucleotides, in each instance, which hybridize on the constant central region of the nucleic acid starting sequence during the production of the first and second partial fragments by means of PCR. The PCR products or the first and second partial fragments are subsequently treated with the USER enzyme, which removes the uracil radical and the nucleotides in the 5′ direction, causing single-strand 3′ overhangs to form. The compatible single-strand ends of the first and second partial fragments are subsequently assembled and, if necessary On the case of short compatible ends) ligated in the presence of a ligase.

In a further embodiment of the method according to the invention for combining the first and second partial fragments, the 3 ends of the first partial fragments overlap with the 5′ ends of the second partial fragments by at least approximately 15, 16, 17, 18, 19 or preferably at least by 20 nucleotides, or the nucleic acid sequence of the 3′ ends of the first partial fragments is homologous or identical with the nucleic acid sequence of the 5′ ends of the second partial fragments by at least approximately 70%, 71%, 72%, 73%, 74%, preferably by at least 75% or 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, and the first and second partial fragments are combined with one another by way of fusion PCR (“overlap extension” PCR). A person skilled in the art knows that if necessary, an overlap of less than 20 nucleotides or identity of slightly less than 75%, in other words e.g. 72 or 73%, can still be sufficient to allow fusion of the partial fragments. This depends, of course, on the composition or the nucleotide composition of the 5′ ends of the second partial fragments and of the 3′ ends of the first partial fragments. The use of fusion PCR for combining partial fragments is described in greater detail in the following documents: Horton et al., 1989, Gene 15:61-8; Horton et al., 1990, Biotechniques 8;528-535, Pogulis et al., 2000, The Nucleic Acid Protocols Handbook Humana Press Inc., Totowa, N.J. 2000 10.1385/1-59259-038-1:857.

The selection of the suitable method for combining the first and second partial fragments depends on different goals and conditions, such as, for example, the length of the nucleic add starting sequence and the length variants derived from it, the length of the constant central region. Thus, when combining them by way of fusion PCR or recombination, a longer overlap region between first and second partial fragments must be taken into consideration than when combining them by way of restriction digestion and ligation.

Finally, the partial fragments or the resulting length variants can be cloned into suitable expression vectors. In a preferred embodiment, this is done by way of restriction digestion of the restriction cut sites appended to the ends of the partial fragments—as described in the example, among other things, and subsequent ligation into the vector linearized by way of TypII or TypIIs digestion. Cloning of the partial fragments into the vector can take place in one or more steps. Thus, for example, first combining the partial fragments to produce length variants can take place, before the length variants are inserted into the target vector. Alternatively, combining the partial fragments with the vector can take place at the same time. A suitable method for simultaneous and directed assembly of at least two fragments into a target vector in a “one step, one pot” reaction is made possible, for example, by what is called “Golden Gate” cloning (Engler C, Kandzia R, Marillonnet S (2008) A One Pot, One Step, Precision Cloning Method with High Throughput Capability. PLoS ONE 3(11): e3647). In this connection, not only the fragments to be inserted but also the vector is digested with TypIIs restriction enzymes, thereby producing specific compatible overhangs at all ends, which can subsequently be jointed together only in a specific combination.

In a further embodiment, the length variants can be introduced into a target vector by means of fusion PCR, if the ends of the length variants have a sufficient overlap region with the ends of a linearized vector. Insertion of fragments into vectors by way of fusion PCR is described, for example, in what is called the CPEC method (“Circular Polymerase Extension Cloning,” Quan J, Tian J (2009) Circular Polymerase Extension Cloning of Complex Gene Libraries and Pathways. PLoS ONE 4(7): e6441). This method allows not only the insertion of individual length variants into a target vector, but also simultaneous insertion of multiple partial fragments by means of overlapping or extensively homologous regions between (i) the outer ends of the partial fragments and the ends of the linearized vector, and (ii) the facing ends of the first and second partial fragments in the region of the constant central regions.

In a further embodiment, the length variants or partial fragments and the vector can also be assembled after production of single-strand overlapping ends on all molecules, using exonucleases, by means of in vitro recombination. A suitable method is, for example, what is called the “SLIC” method, which describes a sequence-independent and ligation-independent cloning method for one-step assembly of multiple molecules, in which, for example, T4 exonuclease is used to generate single-strand overhangs. (Li M Z, Elledge S J. Harnessing homologous recombination in vitro to generate recombinant DNA via SLIC. Nat Methods 2007 Mar;4(3):251-6). Further suitable exonucleases for corresponding methods are mentioned at other points in this document. Hybridization of the single-strand ends can be supported by means of RecA, for example.

Finally, in a further embodiment the length variants or partial fragments can also be cloned into corresponding vectors using Gateway® cloning technology, by means of in vitro recombination by way of att sites. Suitable vectors and methods are known to a person skilled in the art and are described, for example, in U.S. Pat. No. 5,888,732, U.S. Pat. No. 6,143,557, U.S. Pat. No. 6,171,861, U.S. Pat. No. 6,270,969 or U.S. Pat. No. 8,277,608.

The disclosures of the aforementioned documents are hereby incorporated into the present patent by reference. Also, the methods indicated above can be combined with one another, e.g. combining the partial fragments by way of TypIIs digestion and ligation and subsequent recombination of the length variants into Gateway vectors or e.g. combining the partial fragments by way of TypIIs digestion and ligation and subsequent insertion of the length variants into the target vector by means of fusion PCR, etc.

Common vectors are, for example, pET, pBAD, pQE, pTrcHis, pGEX etc. The vectors that contain the length variants can then be introduced, separately or as a defined mixture of vectors or as a fragment library, into a suitable prokaryotic (e.g. Escherichia coli, Bacifius, Caulobacter, Lactobacillus, Lactococcus, Salmonella, Streptomyces) or a eukaryotic expression system (e.g. molds, Baculovirus/plant, yeast, insect or mammalian cells), which produces the shortened polypeptide variants or the polypeptide library. Common expression systems and their advantages and disadvantages are described, for example, in Schirrman et al. 2008, Frontiers in Bioscience 4578-4594 or in Brondyk et al. 2009, Methods in Enzymology, 463:131-147. Subsequently, suitable candidates with desired properties are selected from the polypeptide library, by way of a suitable selection method.

Thus, the method according to the invention also comprises the production of a polypeptide library, characterized by the following steps
a) Introduction of the fragment library into suitable expression vectors,
b) Introduction of the expression vectors into eukaryotic or prokaryotic cells,
c) Cultivation of the cells under conditions that are suitable for allowing expression of the fragment library,
d) and, if necessary, isolation of the polypeptide library,
e) and, if necessary, screening of the polypeptide library into a suitable selection method for identification of one or more candidates with desirable properties.

Alternatively, the fragment library can be expressed in a cell-free system and subsequently purified or isolated. Preferably, this involves an in vitro transcription-translation system. Suitable cell-free systems for protein production, for example from insect cell lysate (Ezure et al., 2010, Curr Pharm Biotechnol. 11:279-84) from “wheat germ” (Takai et al., 2010 Curr Pharm Biotechnol. 11:272-8), from rabbit reticulocytes (Promega) or from E. coli (RTS lysate), as well as synthetic systems such as, for example, the PURESYSTEM® from BioComber (synthetic lysate with E. coli ribosomes), which was developed by Takuya Ueda (Shimizu and Ueda, 2010, Methods Mol Biol. 607:11-21) or systems based on it (e.g. WO2005105994) are known to a person skilled in the art.

Therefore the method according to the invention also comprises the production of a polypeptide library characterized by the following steps
a) Introduction of the fragment library into suitable expression vectors,
b) Incubation of the expression vectors with a suitable in vitro transcription-translation system,
c) if necessary, isolation of the polypeptide library,
d) and, if necessary, screening of the polypeptide library in a suitable selection method for identification of one or more candidates with desired properties.

Using the polypeptide library according to the invention, functional domains or amino add regions can be identified, epitopes can be mapped, inhibitors can be screened or functional shortened signal peptides can be generated and selected. Furthermore, the polypeptide library can be used for screening of enzymes with improved properties, which are suitable, for example—as explained above—for crystallization studies. Furthermore, new functional minimal proteins can be generated and selected.

Furthermore, the fragment libraries according to the invention can also be simply transcribed, if, for example, RNA sequences are supposed to be investigated or screened. For this purpose, a commercial cell-free in vitro transcription system such as the RiboMAX™ “Large Scale RNA Production System-T7” (U.S. Pat. No. 5,256,555, U.S. Pat. No. 6,586,218 and U.S. Pat. No. 6,586,219), for example, can be used.

Thus, the method according to the invention also comprises the production of an RNA library characterized by the following steps
a) Introduction of the fragment library into suitable expression vectors,
b) Incubation of the expression vectors with a suitable in vitro transcription system,
c) if necessary, isolation of the RNA library,
d) and, if necessary, screening of the RNA library in a suitable selection method for identification of one or more candidates with desired properties.

Typical cell-base or virus-based selection method for the polypeptide libraries produced according to the method according to the invention are, for example, Bacterial Display (Kemp, 1981, Proc. Natl. Acad. Sci. USA 78(7): 4520-4524) or bacterial display by way of Bacillus endospores (s. Kim and Schuhmann, 2009, Cell Mol. Life Sri. 66:3127-3136), Yeast Two Hybrid (e.g. described in U.S. Pat. No. 5,283,173), Phagen Display (e.g. Sidhu et al., 2003, Chembiochem 4:14-25; U.S. Pat. No. 5,223,409, U.S. Pat. No. 5,403,484, U.S. Pat. No. 5,571,698, and U.S. Pat. No. 5,837,500), Yeast Surface Display (described e.g. in WO08118476, WO09093118, Chien et al., 1991, Proc Nati Acad Sci USA 88:9578-82; Boder and Wittrup, 1997, Nat. Biotechnol. 15:553-557), or retroviral or lentiviral display methods (e.g. Buchholz et al., 2008, Comb Chem 11:99-110; Taube at al., 2008, PLoS 3:e3181). Alternatively, cell-free selection systems such as, for example, mRNA Display (see e.g. WO98/54312 PROFUSION™, U.S. Pat. No. 6,258,558, U.S. Pat. No. 6,518,018, U.S. Pat. No. 6,281,344, U.S. Pat. No. 6,214,553, U.S. Pat. No. 6,261,804, U.S. Pat. No. 6,207,446, U.S. Pat. No. 6,846,655, U.S. Pat. No. 6,312,927, U.S. Pat. No. 6,602,685, U.S. Pat. No. 6,416,950, U.S. Pat. No. 6,429,300, U.S. Pat. No. 7,078,197, and U.S. Pat. No. 6,436,665), ribosomal display (see e.g. WO0175097, U.S. Pat. No. 5,643,768, U.S. Pat. No. 5,658,754, and U.S. Pat. No. 7,074,557) or microcompartmentalization (Sergeeva at al., 2006, Adv Drug Deliv Rev. 58(15):1622-1654) for identification of improved polypeptide or protein candidates, by means of in vitro transcription/translation. Many display systems known to a person skilled in the art and the application areas, in each instance, are described, for example, in the overview article Seergeva et al., 2006, Adv. Drug Deliv. Rev. 58:1622-1654,

The disclosures of the aforementioned documents are hereby incorporated into the present patent document by reference.

Furthermore, the polypeptide library according to the invention can be used for screening of soluble polypeptide variants. In the state of the art, different methods for identification of soluble protein derivates from corresponding fragment libraries are described. Frequently, the corresponding fragments or variants are fused with a reporter gene, the function of which can be easily detected as a function of the soluble expression and correct folding of the fused target protein or fragment. For example, green fluorescing protein (GFP) (Waldo et al., 2003, Curr Opin Chem Biol. 7:33-8, Coleman et al., 2004, J. Proteome Res., 3:1024-1032), chloramphenicol-acetyl-transferase (CAT) (Maxwell et al., 1999, Protein Science 8:1908-1911), beta-galactosidase (β-Gal) or biotin-carboxyl carrier protein (BCCG) (WO03064656) serve as reporter genes. In order to avoid the side effects of large fusion components, alternatively small regions or fragments of reporter genes are often fused to the target sequence, which, after expression of the fusion construct, are only activated by means of self-complementing with the missing fragment required for the reporter function. Corresponding approaches are described, for example, in Ullmann et al., 1967; Nixon and Benkovic, 2000, Protein Eng. 13(5):323-7; Wigley et al., 2001, Nat Biotechnol. 19:131-6; Wehrman et al., 2002, Proc. Natl. Acad, Sci. U.S.A. 99:3469-74 (complementing of the lacZ fragment with lacZO) or in WO05074436 (complementing of a small fused GFP component with the large GFP fragment). An alternative method for the selection of soluble proteins is disclosed by WO06024875. In this connection, the fragment library is fused with a small peptide substrate, which fuses only in connection with a small peptide substrate, which can only be specifically modified by an enzyme in connection with a soluble fusion partner.

The invention therefore also comprises the use of the described fragment or polypeptide library for screening of soluble polypeptide variants or enzymes, wherein the partial fragments or length variants are cloned into the target vector in fusion with a 3′-located reporter gene, wherein the reporter gene codes for chloramphenicol-acetyl-transferase, for example, and wherein the reporter gene is only detectably expressed if the fused length variant is soluble. Fusion of partial fragments or length variants with the reporter gene can already take place before cloning into the target vector. Alternatively, the reporter gene can already be situated in the target vector, and the fusion takes place by means of cloning of the partial fragments or length variants into the target vector. A possible embodiment of the invention is shown in FIG. 5.

In a particular embodiment, a stop codon (e.g. amber TAG) is introduced between the length variant and the reporter gene. The first screening for solubility then takes place by means of transformation of the length variants ligated into the reporter vector into an amber suppressor strain and placement onto selection plates (e.g. in this embodiment chloramphenicol selection plates). The stop codon in the fusion region of the two fusion partners is read over into a suppressor strain, and translation of a complete fusion protein occurs. Preferably, the chloramphenicol concentration of the plates is selected in such a manner that only colonies that express the soluble fusion products of length variant+chloramphenicol-acetyl-transferase can grow. Alternatively, in addition, differentiation between lame and small colonies can take place, where it is to be expected that large colonies have a tendency to express more soluble fusion protein. In order, in a second step, to verify the solubility of the length variant even without a chloramphenicol-acetyl-transferase component, isolated plasmid-DNA from positively selected colonies can be retransformed into a non-suppressor strain. Here, the amber stop codon always leads to termination of the expression, and the length variant is translated without a fusion component. In this connection, it must be noted that the target selection vector contains a second, chloramphenicol-independent resistance cassette for establishing transformed cells. Verification of the solubility of the length variants expressed in this can thereupon take place by way of usual protein biochemistry methods (e.g. gel chromatography, affinity chromatography, Western Blot, ELISA).

Furthermore, the fragment libraries according to the invention can be used as libraries that have length variants of non-coding or regulatory sequences for screening of functional or regulatory elements (e.g. minimal promoters for improving expression or minimal terminators) or non-regulatory sequence motifs (e.g. untranslated regions, transposons or minimal introns that are spliced well).

### FIGURES

The invention is furthermore explained by means of the following figures.

FIG. 1: Gene sequence of nptII with indication of the coded amino acid sequence using the single-letter code. Start (ATG) and stop (TAA) codons additionally introduced by way of the oligonucleotides are indicated, and the central region, which is kept constant in each length variant, is underlined. The shortening of the nucleic acid starting sequence in 3-codon or 3-amino acid steps is indicated with outlined and non-outlined highlighting of the 5 and 3′ regions. The overhang comprising 4 nucleotides, for combining the first and second partial fragments, is highlighted in black.

FIG. 2: Figure part A shows the production of the first partial fragments of the nptII gene by means of step by step shortening of the 5′ region of the nucleic add starting sequence, using corresponding constructed forward oligonucleotides (SEQ ID NO:1-20), which all contain a start codon and a cut site recognition sequence. Figure part B shows the production of the second partial fragments of the nptII gene by means of step by step shortening of the 3′ region of the nucleic add starting sequence, using corresponding constructed reverse oligonucleotides (SEQ ID NO:21-40), which all contain a stop codon and a cut site recognition sequence. Figure part C shows the counter-oligonucleotides used for both partial fragments (SEQ ID NO:41 and 42), which hybridize, overlapping within the central regions of the nucleic add starting sequence, and contain the TypIIs restriction cut site Bbsl for generating non-palindromic single-strand overhangs, in each instance.

FIG. 3: Oligonucieotides for the production of the partial fragments for the nptII fragment library. The Ndel cut site (CATATG) attached by way of the forward oligonucleotides (SEQ ID NO:1-20) and the cut site for Xhol (CTCGAG) attached by way of the reverse oligonucleotides (SEQ ID NO:21-40), as well as the Bbsl recognition sequences in the counter-oligonucleotides (reverse: SEQ ID NO:41 and forward: SEQ ID NO:42) are framed. The overhang generated by means of restriction with Bbsl is double-underlined.

FIG. 4: Distribution of the nptII length variants within the fragment library: The statistically expected distribution of the nptII reading frame lengths is cross-hatched in white and black, and the distribution of the nptII reading frame lengths actually observed is shown in black.

FIG. 5: Production of a fragment library for identification of soluble polypeptide variants: Based on a nucleic acid starting sequence that codes for a protein or polypeptide to be analyzed, first and second partial fragments are generated after definition of a constant region (cross-hatched, e.g. minimal functional region), using specific oligonucleotides in separate PCR methods, which fragments contain the desired 5 and/or 3′ shortening and half of the constant central region. Each first partial fragment contains a specific TypIIs cut site at the 3′ end, and each second partial fragment contains it at the 5′ end (in the constant central region, in each instance). After restriction digestion, the single-strand overhangs of the first and second partial fragments that are formed are connected to produce length variants (e.g. by means of ligation or recombination). In the embodiment shown, the first partial fragments on the 5′ end and the second partial fragments on the 3′ end were provided, in each instance, with further TypII or TypIIs cut sites, in order to allow subsequent cloning of the length variants obtained into a target vector. The vector contains a reporter gene (in this case a CAT gene), which results in a reporter fusion protein after insertion of the length variant. Optionally, the fusion site can be interrupted by way of an amber stop codon, in order to allow expression of the fusion proteins or the length variant alone, depending on the strain used. The vectors that contain the different length variants can subsequently be introduced into suitable expression systems, such as E. coli, for example, in order to test the expressed, shortened polypeptide variants for solubility, as a function of the expression efficiency.

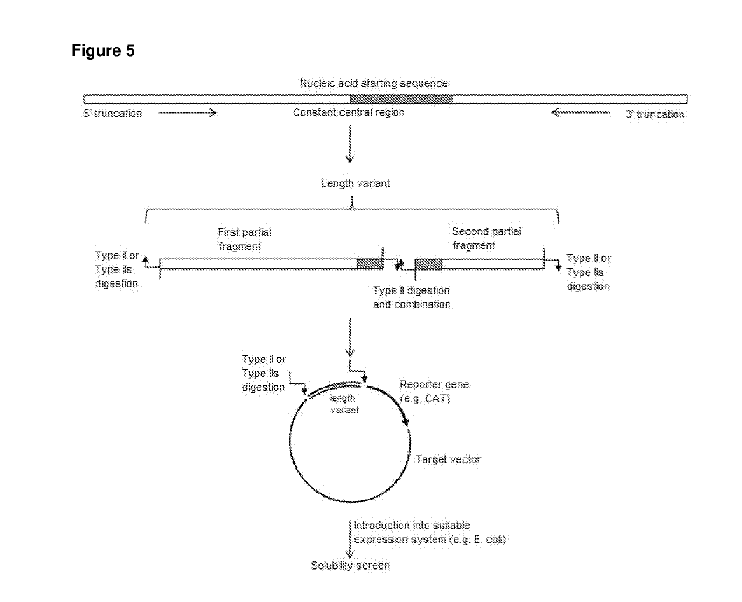

### EXAMPLE

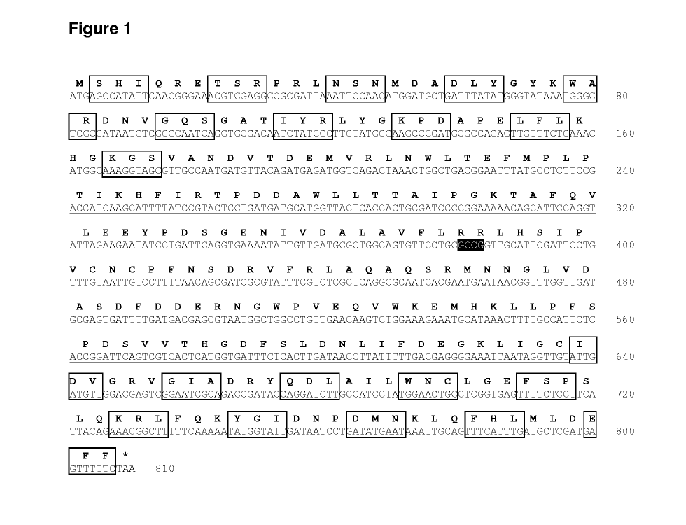

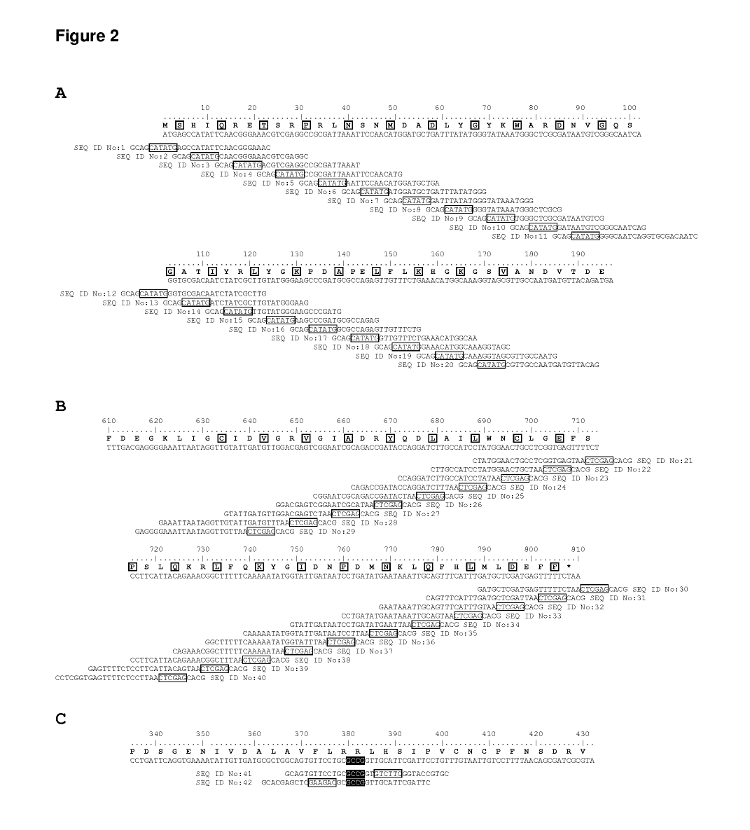

In this exemplary embodiment, the gene nptII (SEQ ID NO:43), which codes for kanamycin-resistance-mediated aminoglycoside-3′-phosphotransferase Type II (SEQ ID NO:44) and served as a nucleic acid starting sequence, was shortened in 2×20 steps of 3 codons (or 3 amino adds) each, from the 5′ and 3′ end, in each instance. (FIG. 1 alternately highlighted in gray/white). Because the combinations of the 20 first partial fragments with the 20 second partial fragments are random, this results in a total number of 20×20 =400 possible combinations. When shortening by 3 codons, in each instance, the start and stop codon (FIG. 1, outlined) are not counted, but instead, appended to each partial fragment, in order to guarantee correct translation even of the shortened length variants. Accordingly, a constant central region of 154 codons (coding for VANDV . . . KLIGC, underlined in FIG. 1) was established for all the length variants. For subsequent combining of the first and second partial fragments, a non-palindromic sequence region von 4 by approximately in the center of the central region was selected (GCCG, highlighted in black in FIGS. 1 and 2, in each instance).

For the production of all 20 first partial fragments, which represent the shortening of the 5′ region of the nucleic add starting sequence, and all 20 second partial fragments, which represent the shortening of the 3′ region of the nucleic add starting sequence, the oligonucleotide pairs were selected in such a manner that it was possible to amplify one of the partial fragments in the desired size with every oligonucleotide pair, by way of PCR. In this connection, during production of the first partial fragments, additionally a start codon as well as the cloning cut site Ndel was appended, in each instance, by way of the oligonucleotides (FIG. 2A), and accordingly, in the production of the second partial fragments, additionally a stop codon as well as the cloning cut site Xhol was appended by way of the oligonucleotides (FIG. 2B). Within the central region, an oligonucleotide was selected for the production of the first and second partial fragments, in each instance, in such a manner that it functioned as a counter-oligonucleotide for the 5′-forward and the 3′-reverse oligonucleotides. These two oligonucleotides (SEQ ID NO:41 and 42), which hybridize with part of the central region, contain the non palindromic sequence region of 4 base pairs for ligation, followed by a Bbsl cut site, in each instance (FIG. 2C). The restriction enzyme Bbsl belongs to the TypIIs enzymes, which cut outside of their recognition sequence. Thus, for example, the recognition sequence for Bbsl is GAAGACNN′XXXX. In this case, XXXX represents the non-palindromic sequence region for ligation (GCCG).

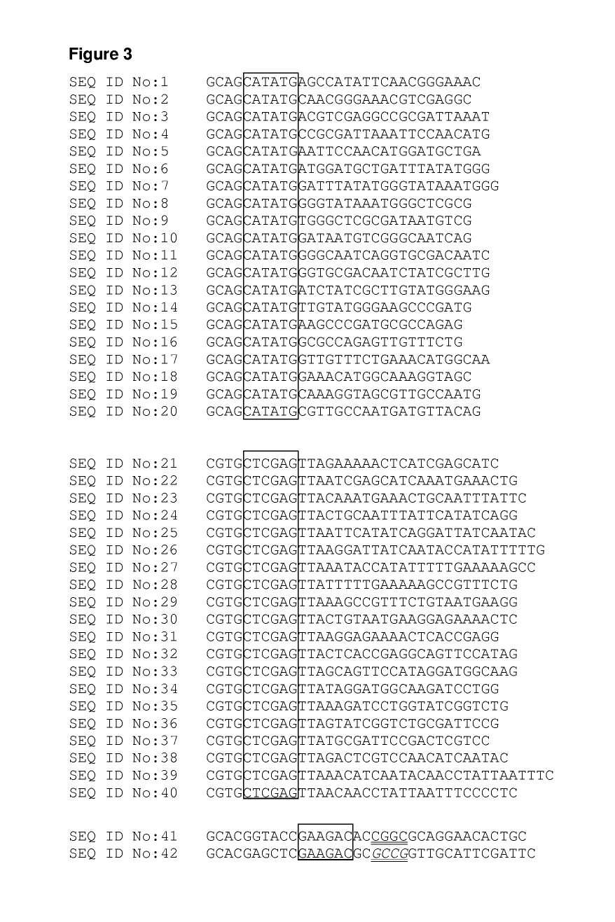

The oligonucleotides required for the production of the first 20 and second 20 partial fragments are listed FIG. 3 (SEQ ID NO:1-42). With these oligonucleotides, 40 PCR reactions were set up according to a standard protocol, according to the plan indicated below (PCR01 to PCR2O for the production of the first partial fragments and PCR21 to PCR40 for the production of the second partial fragments). In each instance, the synthetically produced gene nptII (SEQ ID NO:43) was used as a nucleic acid starting sequence.

PCR01: SEQ ID NO:1 and SEQ ID NO:41

PCR02: SEQ ID NO:2 and SEQ ID NO:41

. . .

PCR20: SEQ ID NO:20 and SEQ ID NO:41

PCR21: SEQ ID NO:42 and SEQ ID NO:21

PCR22: SEQ ID NO:42 and SEQ ID NO:22

. . .

PCR40: SEQ ID NO:42 and SEQ ID NO:40

The PCR products were purified with a silica membrane, using small columns, and subsequently their concentration was determined photometrically. Equal molar amounts were combined, cut with the TypIIs restriction enzyme Bbsl, and purified again. The entire mixture of the digested first and second partial fragments was mixed with ligase and incubated at room temperature for 3 h, where it was possible to combine each first partial fragment with each second partial fragment. Because the overhangs generated by the Bbsl endonuclease are not palindromic, the undesirable combinations of first with first partial fragments and of second with second partial fragments were avoided. The reaction was stopped with a heating step, and the ligated length variants were purified again, subsequently cut with the TypII restriction enzymes Ndel and Xhol, and ligated into the target vector pEG-His1, which had also been cut. The ligation batch was transformed into competent E. coli DH10 B cells and placed onto ampicillin and kanamycin/IPTG selection medium. The vector pEG-His1 is am expression vector that can be induced by IPTG and carries constitutive ampicillin resistance for selection purposes. Accordingly, it could be expected that all the cells that had received a vector from the ligation batch could grow on ampicillin plates. In contrast, only those cells that carried a recombinant vector, the nptII gene of which coded for functional aminoglycoside-3′-phosphotransferase, and in which the corresponding amino-terminal and/or carboxyl-terminal shortened regions did not impair the activity of the protein, were supposed to be kanarnycin-resistant.

Accordingly, random sample analysis of the colonies growing on ampicillin allowed a statement about the neutral overall distribution of the partially shortened nptII genes within the overall fragment library. Random sample analysis of the colonies growing on kanamycin, on the other hand, allowed a functional analysis of the gene bank, in that the minimal size of the active aminonlycoside-3′-phosphotransferase was determined.

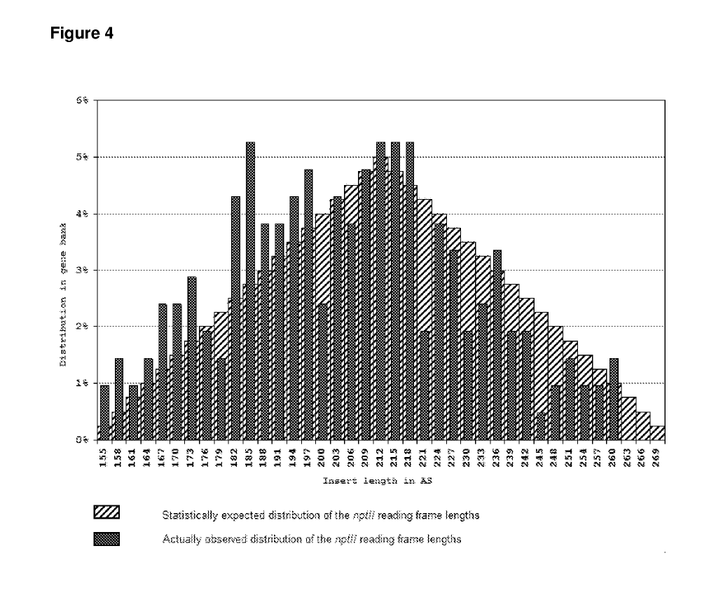

In total, the nptII gene from 209 ampicillin-resistant colonies was sequence-analyzed. The statistical distribution of the total length of the nptII reading frame corresponded to the expected distribution, with the slight restriction that shorter fragments were slightly over-represented (FIG. 4). This is probably due to the higher ligation efficiency of shorter fragments and could be compensated by means of a corresponding increase in the amount of longer first and second partial fragments used in the initial ligation. Separate consideration of the shortening of the 5′ and 3′ regions of the nucleic acid starting sequence showed that all the expected decreases in length were found in the non-selected random sample of 209 units.

A sequence analysis of the kanamycin-resistant colonies yielded the result, for the carboxyl terminus, that the last three amino acids are essential for the functionality of the protein. (A more detailed result on an individual amino acid level could only be obtained in a further experiment, using a corresponding fine-frame fragment library with decreases in length of only one amino acid, in each instance). For the amino terminus, on the other hand, shortening by up to 21 amino adds can be tolerated for activity of the enzyme. In total, the experiment defines the amino add sequence [MGYKWAR . . . MLDEFF] as a minimal motif for aminoglycoside-3′-phosphotransferase NptII. This information can be used to produce functional protein variants with new properties (for example changed solubility), or to shorten the gene for functioning kanamycin-resistance in vectors by 63 nucleotides. The method according to the invention, for the production of fragment libraries, thereby allows increased screening efficiency by means of a greater proportion of correct or functional clones, thereby making it possible to save resources in the screening of optimal candidates.

1. A fragment library comprising at least two, preferably at least four length variants of a nucleic acid starting sequence, characterized in that
a) each length variant comprises a constant central region and a 5′ and a 3′ region of variable length, in each instance,
b) each length variant is composed at least of a first partial fragment and a second partial fragment, wherein each first partial fragment comprises at least one defined 5′ partial region of the constant central region and each second partial fragment comprises at least one defined 3′ partial region of the constant central region.

2. The fragment library according to claim 1, characterized in that the length variants are composed only of clearly defined first and second partial fragments.

3. The fragment library according claim 2, characterized in that the first and second partial fragments do not demonstrate any reading frame mutations as compared with the nucleic acid starting sequence.

4. The fragment library according to claim 1, wherein at least one part of the nucleic acid starting sequence codes for an amino acid sequence, and the amino acid sequence is defined by at least one central region and one amino-terminal and/or one carboxyl-terminal region, characterized in that each length variant codes for at least one constant central region and an amino-terminal and/or carboxyl-terminal region of variable length, in each instance.

5. The fragment library according to claim 1, wherein at least one part of the nucleic acid starting sequence codes for a polypeptide, a protein, a protein derivative, a protein fragment, a polymer or a fusion protein.

6. The fragment library according to claim 1, wherein at least one part of the nucleic acid starting sequence codes for a functional RNA or a ribozyme.

7. The fragment library according to claim 1, wherein the nucleic acid starting sequence comprises an untranslated regulatory or a non-regulatory sequence.

8. The fragment library according to claim 1, characterized in that the length variants, as compared with the nucleic acid starting sequence, have
a) a shortened 5′ region or
b) a shortened 3′ region or
c) a shortened 5′ region and a shortened 3′ region.

9. The fragment library according to claim 1, characterized in that the length variants, as compared with the nucleic acid starting sequence, have
a) a lengthened 5′ region or
b) a lengthened 3′ region or
c) a lengthened 5′ region and a lengthened 3′ region.

10. The fragment library according to claim 1, characterized in that the length variants, as compared with the nucleic acid starting sequence, have
a) a lengthened 5′ region and a shortened 3′ region or
b) a shortened 5′ region and a lengthened 3′ region.

11. (canceled)

12. The fragment library according to claim 1, characterized in that the length variants, as compared with the nucleic acid starting sequence, comprise additional regulatory sequences selected from restriction cut sites, start and stop codons, polyadenylation signal sequences, promoter or terminator sequences.

13. The fragment library according to claim 1, characterized in that the length variants, as compared with the nucleic acid starting sequence, additionally have insertions or deletions or point mutations within the first or second partial fragments.

14. (canceled)

15. (canceled)

16. A method for the production of a fragment library according to claim 1, comprising at least two, preferably at least four length variants of a nucleic acid starting sequence, characterized in that
a) each length variant comprises a constant central region and, in each instance, a 5′ and a 3′ region of variable length,
b) each length variant is composed at least of a first partial fragment and a second partial fragment, wherein each first partial fragment comprises at least one defined 5′ partial region of the constant central region and each second partial fragment comprises at least one defined 3′ partial region of the constant central region,
c) wherein all the first and second partial fragments are connected with one another, in a step, in such a manner that the length variants have all the possible combinations of first and second partial fragments.

17. The method for the production of a fragment library according to claim 16, wherein furthermore, no combination von first partial fragments with one another or second partial fragments with one another can take place.

18. The method for the production of a fragment library according to claim 16, wherein the 3′ ends of the first partial fragments overlap with the 5′ ends of the second partial fragments by at least approximately 20 nucleotides, or the nucleic acid sequence of the 3′ ends of the first partial fragments is identical with the nucleic acid sequence of the 5′ ends of the second partial fragments by at least approximately 70%, preferably at least 75%, and the first partial fragments are connected with the second partial fragments by way of fusion PCR.

19. The method for the production of a fragment library according to claim 16, wherein the 3′ ends of the first partial fragments and the 5′ ends of the second partial fragments have single-strand overhangs, and the first partial fragments are connected with the second partial fragments by means of ligation.

20. The method for the production of a fragment library according to claim 19, wherein the single-strand overhangs of the first and second partial fragments are produced by means of digestion with an enzyme.

21. The method for the production of a fragment library according to claim 16, characterized in that the production of the partial fragments takes place by means of lengthening of oligonucleotides homologous to a partial region of the nucleic acid starting sequence, by means of a PCR-based method (primer extension).

22. The method for the production of a fragment library according to claim 16, wherein the production of the first partial fragments is characterized by the following steps
a) Making available a defined nucleic acid starting sequence,
b) Making available a first oligonucleotide for the synthesis of a first partial fragment, wherein the 3′ region of the first oligonucleotide is identical with a defined nucleic acid sequence in the 5′ region of the nucleic acid starting sequence by at least approximately 70%, preferably at least 75%,
c) Making available a second oligonucleotide, wherein the 3′ region of the second oligonucleotide is identical with a defined nucleic acid sequence of the constant central region of the nucleic acid starting sequence by at least approximately 70%, preferably at least 75%,
d) Incubation of the nucleic acid starting sequence from (a) with the first oligonucleotide from (b), the second oligonucleotide from (c) and a polymerase system under conditions that are suitable for allowing hybridization of the homologous 3′ regions of the oligonucleotides at single-strand regions of the nucleic acid starting sequence and lengthening of the hybridized oligonucleotides by means of polymerase chain reaction,
e) Making available a third oligonucleotide for the synthesis of a second 5′-partial fragment, wherein the 3′ region of the third oligonucleotide is identical with a defined nucleic acid sequence in the 5′ region of the nucleic acid starting sequence by at least approximately 70%, preferably at least 75%, and differs from the 3′ regions of the first oligonucleotides in at least one, preferably in at least three nucleotides,
f) Incubation of the nucleic acid starting sequence from (a) with the third oligonucleotide from (e), the second oligonucleotide from (c) and a polymerase system under conditions that are suitable for allowing hybridization of the homologous 3′ regions of the oligonucleotides to individual-strand regions of the nucleic acid starting sequence and lengthening of the hybridized oligonucleotides by means of polymerase chain reaction,
g) if necessary, making available further oligonucleotides for the synthesis of further 5′ partial fragments, wherein each 3′ region of each further oligonucleotide is identical with another defined nucleic acid sequence in the 5′ region of the nucleic acid starting sequence by at least approximately 70%, preferably at least 75%, and differs from the 3′ regions of the first and third oligonucleotide in at least one, preferably in at least three nucleotides,
h) Incubation of the nucleic acid starting sequence from (a) with a further oligonucleotide from (g), in each instance, the second oligonucleotide from (c) and a polymerase system, under conditions that are suitable for allowing hybridization of the homologous 3′ regions of the oligonucleotides to the nucleic acid starting sequence and its lengthening by means of polymerase chain reaction.
Repetition of steps (g) and (h) until all the desired 5′ partial fragments have been synthesized.

23. The method for the production of a fragment library according to claim 16, wherein the production of the second partial fragments is characterized by the following steps
a) Making available a defined nucleic acid starting sequence,
b) Making available a first oligonucleotide for the synthesis of a first 3′ partial fragment, wherein the 3′ region of the first oligonucleotide is identical with a defined nucleic acid sequence in the 3′ region of the nucleic acid starting sequence by at least approximately 70%, preferably at least 75%,
c) Making available a second oligonucleotide, wherein the 3′ region of the second oligonucleotide is identical with a defined nucleic acid sequence of the constant central region of the nucleic acid starting sequence by at least approximately 70%, preferably at least 75%,
d) Incubation of the nucleic acid starting sequence from (a) with the first oligonucleotide from (b), the second oligonucleotide from (c) and a polymerase system under conditions that are suitable for allowing hybridization of the homologous 3′ regions of the oligonucleotides at single-strand regions of the nucleic acid starting sequence and lengthening of the hybridized oligonucleotides by means of polymerase chain reaction,
e) Making available a third oligonucleotide for the synthesis of a second 3′ partial fragment, wherein the 3′ region of the third oligonucleotide is identical with a defined nucleic acid sequence in the 3′ region of the nucleic acid starting sequence by at least approximately 70%, preferably at least 75%, and differs from the 3′ regions of the first oligonucleotide in at least one, preferably three nucleotides,
f) Incubation of the nucleic acid starting sequence from (a) with the third oligonucleotide from (e), the second oligonucleotide from (c) and a polymerase system under conditions that are suitable for allowing hybridization of the homologous 3′ regions of the oligonucleotides to single-strand regions of the nucleic acid starting sequence and lengthening of the hybridized oligonucleotides by means of polymerase chain reaction,
g) if necessary, making available further oligonucleotides for the synthesis of further 3′ partial fragments, wherein each 3′ region of each further oligonucleotide is identical with another defined nucleic acid sequence in the 3′ region of the nucleic acid starting sequence by at least approximately 70%, preferably at least 75%, and differs from the 3′ regions of the first and third oligonucleotide in at least one, preferably at least three nucleotides,
h) Incubation of the nucleic acid starting sequence from (a) with a further oligonucleotide from (g), in each instance, the second oligonucleotide from (c) and a polymerase system under conditions that are suitable for allowing hybridization of the homologous 3′ regions of the oligonucleotides to the nucleic acid starting sequence and its lengthening by means of polymerase chain reaction.
Repetition of steps (g) and (h), until all the desired 3′ partial fragments have been synthesized.

24-30. (canceled)
